(19) United States
(12) Patent Application Publication
(10) Pub. No.: US 2008/0172042 A1
(43) Pub. Date: Jul. 17, 2008

(54) Urinary catheterization assembly with open ended sheath

(76) Inventor: Jamie Glen House, Colorado Springs, CO (US)

Correspondence Address: MOAZZAM & ASSOCIATES, LLC, 7601 LEWINSVILLE ROAD, SUITE 304, MCLEAN, VA, 22102, US

(21) Appl. No.: 11/652,526

(22) Filed: Jan. 12, 2007

(51) Int. Cl. A61M 27/00; A61M 3/00

(52) U.S. Cl. 604/544; 604/43

(57) ABSTRACT

A urinary catheter assembly is disclosed having a catheter, a sheath enclosing an insertable portion of the catheter, and a free end partially open to allow fluid communication between the lumen of the sheath and an environment outside the sheath.

### BACKGROUND OF THE INVENTION

1. Field of the Invention

The present invention is directed to devices and methods for catheterization of the urinary bladder. More particularly, the present invention is directed to such devices and methods employing a sheathed catheter.

2. Background of the Invention

The occasional or periodic catheterization of an individual's urinary bladder is a common practice today for many persons who are in a hospital setting, a nursing home, doctor's office, rehabilitation facility or at home. For instance, a patient may be catheterized to treat such conditions as urinary retention, the inability to evacuate urine, or for the purpose of obtaining a sterile urine specimen from a patient in a doctor's office.

Generally, catheterizations may be assisted (non self-catheterizations) or unassisted (self-catheterizations). In assisted catheterizations, a physician or nurse may perform the catheterization by employing a catheterization tray (“cath tray”), which typically includes a sterile drape, gloves, a conventional catheter, antiseptic solution, cotton balls, lubricant, forceps, underpad and a urine collection container. All of these items are typically packaged together and sterilized. However, to perform the catheterization, the nurse must open the tray and handle the various items in the tray. Although special precautions may be taken, maintaining a sterile environment during the procedure may present challenges. In addition, because multiple steps are involved in the procedure, a nurse may spend a significant amount of time (e.g., 10-15 minutes) carrying out each catheterization. Still further, conventional “cath tray” procedures may be expensive or otherwise impractical for use with some individuals and situations today.

For some of the above-stated reasons, many individuals, if capable, would prefer to perform self-catheterization. For many, the level of risk and discomfort of repeated catheterizations carried out over the course of a day (e.g., at 3-6 hour intervals) are offset by the accompanying convenience, privacy or self-reliance that is achieved. Some of the major difficulties that arise in self-catheterization are problems relating to maintaining the required level of sanitation during the procedure, and the difficulty of sometimes performing the procedure under conditions of restricted space and privacy.

A variety of catheterization kits and products are currently available. Some catheter assemblies include a protective sheath that surrounds the catheter or a portion thereof. For instance, U.S. Pat. No. 6,090,075 (House I) discloses a catheter assembly including a catheter introducer member for positioning the catheter assembly against the urethral opening, a flexible catheter, and a pliable thin-walled sheath surrounding the catheter and coupled to the catheter introducer. Such a protective sheath may serve multiple purposes. For instance, the sheath may prevent contamination of the catheter as the catheter is handled prior to and/or during use. In addition, when used with some hydrophilic catheters, the sheath may contain a wetting agent that is used to lubricate the catheter. Failure of the sheath may lead to various problems, such as urinary tract infections, pain, and/or difficult catheterizations.

In some catheter assemblies that include a protective sheath enclosing the catheter, the sheath is gathered near the distal end of the catheter (i.e., the end of the catheter distal the patient) as the catheter is advanced from the protective sheath and into the patient's urethra. As the sheath gathers, the volume within the sheath lumen may decrease. If the sheath lumen is not in fluid communication with the environment outside the sheath, some fluid contained within the sheath lumen (e.g., air, wetting agent, etc.) may become trapped and compressed, potentially resulting in a pressure buildup within the sheath (e.g., the sheath may “balloon-up”). A slight to moderate pressure build-up within the sheath may not cause any problems, however, an excessive pressure build-up within the sheath may result in significant problems. For example, an excessive pressure buildup within the sheath may limit the degree to which the catheter may be advanced beyond the sheath and into the patient's urethra. For instance, pressure build-up within the sheath may result in forces that generally oppose the continued gathering of the sheath. In addition, an excessive pressure build-up may cause the sheath to “balloon-up,” potentially limiting the ability of the user (e.g., physician, nurse, individual, etc.) to adequately grasp portions of the catheter contained within the sheath. In some extreme cases, an excessive pressure build-up within the sheath may result in a tear or rupture of the protective sheath, potentially exposing the catheter contained within the torn sheath to contaminated or non-sterile surfaces. In instances in which excessive pressure-build-up within the sheath may arise, it would be advantageous to have a mechanism or means to relieve some of the pressure within the sheath.

In addition, in some catheter assemblies including a protective sheath enclosing the catheter, the distal end of the sheath (i.e., end of the sheath distal the patient) is physically connected to the distal end of the catheter. From a manufacturing perspective, the process of attaching the distal end of the sheath to the distal end of the catheter may require additional materials (e.g., a collar to couple to the sheath to the catheter), additional steps (e.g., heat sealing the sheath to the catheter), and associated manufacturing costs.

Accordingly, there remains a need for an easy-to-use, sanitary and disposable catheterization assembly. Such an assembly would be well received if it includes a flexible sheath that protects the catheter from contamination, that may be easily gathered without an excessive pressure build-up within the sheath, and is relatively simple and inexpensive to manufacture.

### SUMMARY OF THE INVENTION

These and other needs in the art are addressed in one embodiment by a urinary catheter assembly. In an embodiment, the catheter assembly comprises a catheter including a first end having a urine inlet, a second end having a urine outlet, and a urethra-insertable portion; an introducer adapted to slidingly engage the catheter, wherein the first end of the catheter is at least partially disposed within the introducer; and a pliable sheath comprising a lumen, an introducer end coupled to the introducer, and a free end opposite the introducer end, the introducer and sheath together enclosing all of the urethra-insertable portion; wherein the free end includes an opening in fluid communication with the lumen and an environment outside the lumen; wherein the opening has a size sufficient to permit the second end of the catheter to pass therethrough.

The foregoing has broadly outlined certain features of the embodiments described herein in order that the detailed description that follows may be better understood. Additional features will be described hereinafter. It should be appreciated by those skilled in the art that the conception and the specific embodiments disclosed may be readily utilized as a basis for modifying or designing other devices, methods, or systems for carrying out the same purposes of the embodiments disclosed herein. It should also be realized by those skilled in the art that such equivalent constructions do not depart from the spirit and scope of the invention as set forth in the appended claims.

### BRIEF DESCRIPTION OF THE DRAWINGS

FIG. 1 is a side view of an embodiment of a sheathed catheter assembly.

FIG. 2 is a side view of the sheathed catheter assembly of FIG. 1 with the sheath partially gathered.

FIG. 3 is a side view of the sheathed catheter assembly of FIG. 1, with the sheath substantially gathered.

FIG. 4 is an end view of the sheathed catheter assembly of FIG. 2.

FIG. 5 is a side view of an embodiment of a sheathed catheter assembly.

FIG. 6 is a side view of the sheathed catheter assembly of FIG. 5, with the sheath substantially gathered.

FIG. 7 is a partial side view of a sheathed catheter assembly of FIG. 3 including a urine collection receptacle.

FIG. 8 is a partial side view of a sheathed catheter assembly of FIG. 3 coupled to a urine collection receptacle.

FIG. 9 shows the contents of a catheterization kit according to an embodiment of the present invention.

### DETAILED DESCRIPTION OF THE INVENTION

The following discussion is directed to various embodiments of the invention. The embodiments disclosed herein should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Certain terms are used throughout the following description and claims to refer to particular system components. As one skilled in the art will appreciate, different persons may refer to a component by different names. This document does not intend to distinguish between components that differ in name but not function. The drawing figures are not necessarily to scale. Certain features of the invention may be shown exaggerated in scale or in somewhat schematic form and some details of conventional elements may not be shown in interest of clarity and conciseness.

In the following discussion and in the claims, the terms “including” and “comprising” are used in an open-ended fashion, and thus should be interpreted to mean “including, but not limited to . . . . ” Also, the term “couple” or “couples” is intended to mean either an indirect or direct connection. Thus, if a first device couples to a second device, that connection may be through a direct connection, or through an indirect connection via other devices, components, and connections.

In the following discussion and in the claims, the terms “distal” and “proximal” refer to positions or locations relative to a patient when the catheter assembly is in use. Thus, as used herein, the term “distal” refers to positions or locations relatively away from the patient, while the term “proximal” refers to positions or locations relatively near the patient. For example, the distal end of a catheter is the end of catheter furthest from the patient when the catheter is in use, and the proximal end of the catheter is the end of catheter nearest the patient when the catheter is in use.

U.S. Pat. No. 6,090,075 (House I), U.S. App. No. 60/708,893 (House II), and U.S. application Ser. No. 11/326,699 (House III), and U.S. App. No. 60/781,225 (House IV) are each hereby incorporated herein by reference in their entirety.

Referring to FIG. 1, an embodiment of a sheathed catheter assembly 100 is shown. Assembly 100 comprises a flexible catheter 10 and a protective sheath 40. Sheath 40 encloses substantially all, or at least part of, catheter 10, thereby resulting in an internal space or lumen 41 between sheath 40 and catheter 10. Assembly 100 generally has a center line or axis 50.

Catheter 10 comprises a proximal or first end 11 and a distal or second end 16. First end 11 includes a tip 12 and one or more urine inlet(s) 13. Second end 16 includes a urine outlet 17. A through bore in catheter 10 permits fluid communication between inlet(s) 13 and outlet 17. Thus, when sufficiently inserted into a patient's bladder, urine drains from the patient's bladder into inlet(s) 13, through the bore of catheter 10, and exits catheter 10 at outlet 17.

Catheter 10 may also be described as including a urethral insertable portion 20 and a non-insertable portion 30. Insertable portion 20 of catheter 10 is the length of catheter 10 that may be appropriately inserted into the patient's urethra in order to perform a successful urinary catheterization. Non-insertable portion 30 of catheter 10 is the length of catheter 10 generally not intended to be inserted into the patient's urthra. Urethra-insertable portion 20 extends from first end 11 to an insertion stop location 15, generally defining the intersection of insertable portion 20 and non-insertable portion 30. Non-insertable portion 30 extends from insertion stop location 15 to second end 16. The length of insertable portion 20 is preferably about ⅔ the total length of catheter 10, where the total length of catheter 10 is generally the distance from first end 11 to second end 16. Thus, the length of non-insertable portion 30 is preferably about ⅓ the total length of catheter 10.

The actual length of catheter 10, and hence the actual length of insertable portion 20 and non-insertable portion 30, depends on a variety of factors including without limitation, the application of catheter 10 (e.g., adult or child size), the patient's anatomy (e.g., length of the patient's urethra, etc), or combinations thereof. As a practical matter, it should be understood that the length of insertable portion 20 is decreased, at least to some extent, by any distance on catheter 10 required to accommodate introducer 60 (if provided) and sheath 40 when sheath 40 is gathered proximal second end 16 of catheter 10 as will be described in more detail below.

When catheter 10 is sufficiently inserted into the patient's bladder to drain urine, second end 16 of catheter 10 is controlled and manipulated by the user of catheter 10 (e.g., physician, nurse, individual, etc.) in order to direct the flow of urine exiting outlet 17. In the embodiment illustrated in FIG. 1, second end 16 of catheter 10 is flared such that second end 16 has an increased diameter relative to insertable portion 20 of catheter 10. However, in general, second end 16 may have any suitable geometry including without limitation, a flared or increased diameter geometry (e.g., FIG. 1), a uniform diameter geometry, or a decreased diameter geometry.

Catheter 10 may comprise any flexible catheter. For instance, catheter 10 may be a conventional flexible catheter known in the art. In addition, catheter 10 may comprise any suitable flexible material including without limitation, vinyl, red rubber latex, silicone elastomer, or the like.

Optionally, a urine collection receptacle (e.g., urine collection tray, bag, etc.) may be pre-attached (permanently or removably pre-attached) or may be attached by the user of catheter assembly 100 (e.g., physician, nurse, patient, or other individual) to second end 16 such that outlet 17 is in fluid communication with such a collection receptacle.

Still referring to FIG. 1, first end 11 and catheter tip 12 initially rest inside, and are supported by, an introducer 60. Introducer 60 slidingly engages catheter 10 and guides catheter 10 as insertable portion 20 is advanced through introducer 60 and into the patient's urethra as will be described in more detail below. Introducer 60 may be a similar to those that are known in the art. For example, in some embodiments, introducer 60 may similar to those disclosed in House II. In other embodiments, introducer 60 may be replaced with a protective catheter tip cover or cap employed to keep at least insertable portion 20 of the catheter 10 sterile prior to and during use. Some examples of potential introducers are disclosed in House I, House II, and House III.

As previously discussed, first end 11 is initially disposed within and supported by introducer 60. The contact, and resulting frictional forces, between introducer 60 and catheter 10 preferably maintain first end 11 within introducer 60 prior to use of assembly 100, thereby preventing catheter 10 from inadvertently sliding out of or retracting from introducer 60 into lumen 41 and potentially falling out of sheath 40 through an opening 49 provided in distal end 42 of sheath 40. In some embodiments, an additional membrane (not shown) may be included within introducer 60 to enhance the frictional forces between first end 11 and introducer 60. For instance, catheter 10 may be initially disposed in introducer 60 through a perforation or slit in a silicone (or the like) membrane provided in introducer 60. Such a membrane preferably “grips” catheter 10, thereby enhancing the ability of introducer 60 to maintain first end 11 within introducer 60, and hence prevent catheter 10 from retracting from introducer 60 into lumen 41. In other embodiments, a mechanical means may be included in introducer 60 to permit only one-way axial movement catheter 10 through introducer 60. In still other embodiments, a tubular guide structure (not shown) may extend from introducer 60 into lumen 41 and serve as a means to properly guide tip 12 into introducer 60 in case first end 11 inadvertently retracts from introducer 60 into lumen 41. Although it is desirable to initially maintain first end 11 of catheter 10 within introducer 60, it is also preferable that any forces maintaining first end 11 within introducer 60 may be relatively easily overcome by the user during use of assembly 100 as the user (e.g., physician, patient, nurse, etc.) physically advances tip 12 and insertable portion 20 through introducer 60.

Still referring to FIG. 1, since insertable portion 20 of catheter 10 is inserted into a patient's urethra, at least tip 12, and preferably all, of insertable portion 20, are sufficiently lubricated prior to insertion. Thus, introducer 60 preferably includes a reservoir (not shown) containing a lubricant through which tip 12 and insertable portion 20 pass as insertable portion 20 is advanced through introducer 60 and into the patient's urethra. House I and House II disclose a variety of catheter introducers and tips capable of providing a lubricant to catheter 10. In some embodiments, the lubricant provided within the reservoir in introducer 60 comprises a suitable sticky or tacky lubricating substance that helps to maintain first end 11 within introducer 60 prior to use of catheter assembly 100. Alternatively, or in addition to providing a lubricant in introducer 60, insertable portion 20 may be pre-lubricated. In such pre-lubricated embodiments, introducer 60 and sheath 40 enclosing insertable portion 20 serve to maintain the lubricant on insertable portion 20 and prevent the lubricant from directly contacting surfaces outside sheath 40 and introducer 60.

Still referring to FIG. 1, sheath 40 encloses substantially all of catheter 10 resulting in a space or lumen 41 between sheath 40 and catheter 10. Specifically, sheath 40 encloses substantially all of catheter 10 except those portions resting within introducer 60. Sheath 40 comprises an introducer end 44 coupled to introducer 60 and a free end 42 having an opening 49. Introducer end 44 of sheath 40 may be coupled to introducer 60 by any suitable means including without limitation, via a collar, with an adhesive, by heat pressing or sealing, or combinations thereof. Free end 42 is not coupled to catheter 10 and is permitted to move axially relative to catheter 10.

In the embodiment illustrated in FIG. 1, sheath 40 extends beyond second end 16 of catheter 10 by an extension length 45. Extension length 45 is preferably less than six inches in length, and more preferably between two and three inches. Since sheath 40 extends beyond second end 16, together introducer 60 and sheath 40 completely enclose catheter 10, thereby protecting catheter 10 from contamination prior to and during use of assembly 100.

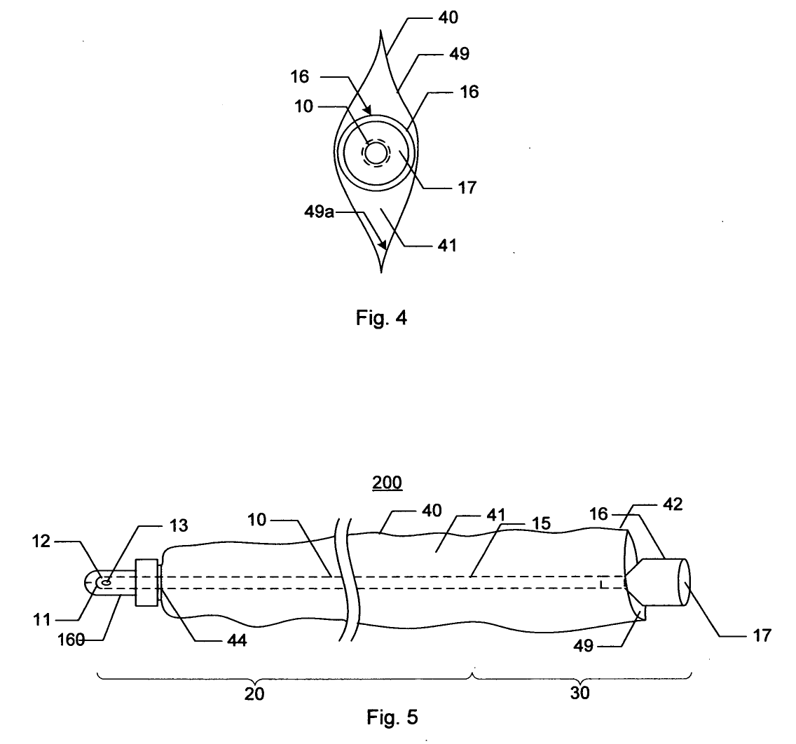

Referring briefly to FIG. 4, opening 49 is provided in free end 42 of sheath 40. Opening 49 permits fluid communication between lumen 41 and the environment outside sheath 40. Opening 49 is sized to permit second end 16 to slide or pass completely through opening 49. Specifically, as shown in FIG. 4, inner surface 49a of opening 49 has an inner surface distance measured substantially perpendicular to catheter 10 that is greater than the perimeter around the outer surface 16a of second end 16 of catheter 10. Without being limited by theory, since sheath 40 comprises a pliable material, regardless of the shape or geometry of second end 16 and opening 49, as long as the inner surface distance of inner surface 49a of opening 49 is greater than the perimeter around the outer surface 16a of second end 16, second end 16 may pass through opening 49. As used herein, the term “inner surface distance” may be used to refer to a distance measured along the inside surface of a sheath substantially perpendicular to a catheter enclosed within the sheath.

In the embodiment illustrated in FIGS. 1-4, sheath 40 has substantially the same inner surface distance along its length. For instance, inner surface 49a of opening 49 has substantially the same inner surface distance as any other portion of sheath 40. In different embodiments (not illustrated), free end 42 may be tapered (e.g., converging) such that opening 49 has an inner surface distance less than the remainder of sheath 40, yet still permit second end 16 to pass completely through opening 49. In still other embodiments (not illustrated), free end 42 may be slightly tapered (e.g., diverging) such that opening 49 has an inner surface distance greater than the remainder of sheath 40.

In addition, it should be understood that no additional components (e.g., collars, etc.) or materials are coupled to free end 42 or used to create opening 49. For instance, opening 49 may be created by simply cutting across sheath 40 during manufacturing. Such a simplistic design of free end 42 and opening 49 may result in a reduction in the manufacturing cost of sheath 40 and assembly 100. For instance, by reducing the use of additional materials, additional components (e.g., collars, etc.), and associated manufacturing steps (e.g., heat sealing a collar to free end 42, coupling free end 42 to second end 16 of catheter 10, etc.), embodiments described herein may beneficially reduce the steps required to manufacture assembly 100 and reduce the manufacturing cost of assembly 100.

Sheath 40 may comprise any suitable pliable material. In select embodiments, sheath 40 is an elongated, thin-walled, flexible plastic bag. Further, sheath 40, and resulting lumen 41, may comprise any suitable geometry including without limitation, rectangular, cylindrical, flattened, inflated, partially inflated, etc.

General Use of the Catheter Assembly

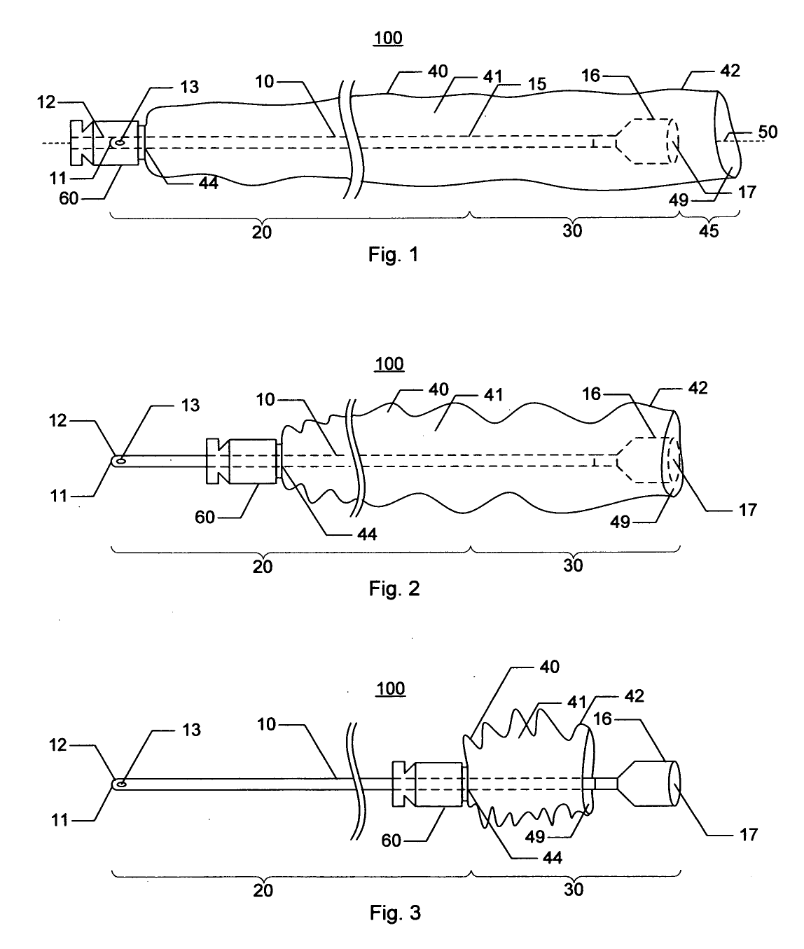

Referring now to FIGS. 1-3, during use, introducer 60 is properly aligned with the patient's urethra. Tip 12 is pushed through introducer 60 to advance tip 12 and insertable portion 20 into the patient's urethra. Introducer 60 slidingly engages catheter 10 and thus catheter 10 moves relative to introducer 60 as insertable portion 20 is advanced through introducer 60. As insertable portion 20 is advanced into the patient's urethra, introducer 60 and introducer end 44 of sheath 40 move generally towards second end 16 of catheter 10, resulting in some gathering of pliable sheath 40 as shown in FIG. 2. At about the same time, free end 42 of sheath 40 extending beyond second end 16 of catheter 10 may also be gathered generally towards second end 16 of catheter 10 giving catheter assembly 100 an appearance similar to that shown in FIG. 2. Continued advancement of insertable portion 20 coupled with continued gathering of sheath 40 gives catheter assembly 100 an appearance similar to that shown in FIG. 3.

In some conventional sheathed catheter assemblies, the process of gathering the sheath while advancing a catheter into the patient's urethra may result in a pressure increase (e.g., a pressure build-up) within the sheath lumen. This may particularly be a problem in sheathed catheter assemblies in which the sheath lumen (e.g., volume within the sheath) is not in fluid communication with an environment outside the sheath. Without being limited by theory, in such sheathed catheter assemblies, as the sheath is gathered, the volume within the lumen may decrease. Consequently, the fluid (e.g., air) within the sheath lumen may become trapped and compressed. The compression of the contents of the lumen may result in a pressure increase within the lumen relative to the environment outside the sheath (e.g., the sheath may “balloon-up” as it is gathered). As previously discussed, pressure build-up within such a protective sheath may result in a variety of problems. For instance, a pressure build-up in the sheath lumen may detrimentally affect the ability of the user to effectively advance the catheter into the patient's urethra (e.g., pressure build-up in the lumen may result in forces tending to oppose the continued advancement of the catheter). In addition, a pressure build-up within the lumen may undesirably reduce the users ability to sufficiently grasp the catheter through the sheath. For example, a pressure build-up in the lumen may result in some expansion (e.g., increase in the diameter) of the lumen, which may render the catheter contained within the lumen somewhat more challenging to handle, position, and/or manipulate.

However, by including opening 49 in the free end 42 of sheath 40 as described herein, fluid communication between lumen 41 and the environment outside sheath 40 is permitted. Thus, any pressure build-up within lumen 41 resulting from the gathering of sheath 40 is relieved through opening 49. In this sense, opening 49 serves as a vent in sheath 40. In other embodiments (not illustrated), one or more additional vents in fluid communication with lumen 41 and the environment outside lumen 41 may also be included in any suitable location including without limitation in sheath 40, introducer 60, or combinations thereof. Some examples of such additional vents are described in House IV.

Use of catheter assembly 100 is preferably conducted in a sterile field, with the user preferably donning sterile gloves to handle assembly 100, thereby reducing the potential for contamination of catheter 10 and urinary tract infections. Protective sheath 40 provides another level of protection to catheter 10 in the event that assembly 100 is used in a non-sterile field or handled with non-sterile surfaces. Although opening 49 in sheath 40 provides some direct physical access to catheter 10, opening 49 in free end 42 of sheath 40 is particularly positioned to minimize the potential for contamination of insertable portion 20. Specifically, opening 49 is positioned distal insertable portion 20 such that any contamination of catheter 10 via opening 49 preferably occurs as far as possible from insertable portion 20, since more distal portion of catheter 10 are least likely to contact the patient or be inserted into the patient's urethra. Thus, it should be appreciated that with opening 49 positioned in free end 42 furthest from insertable portion 20, any direct physical contact with catheter 10 through opening 49 will likely be limited to contact with non-insertable portion 30 of catheter 10, particularly second end 16.

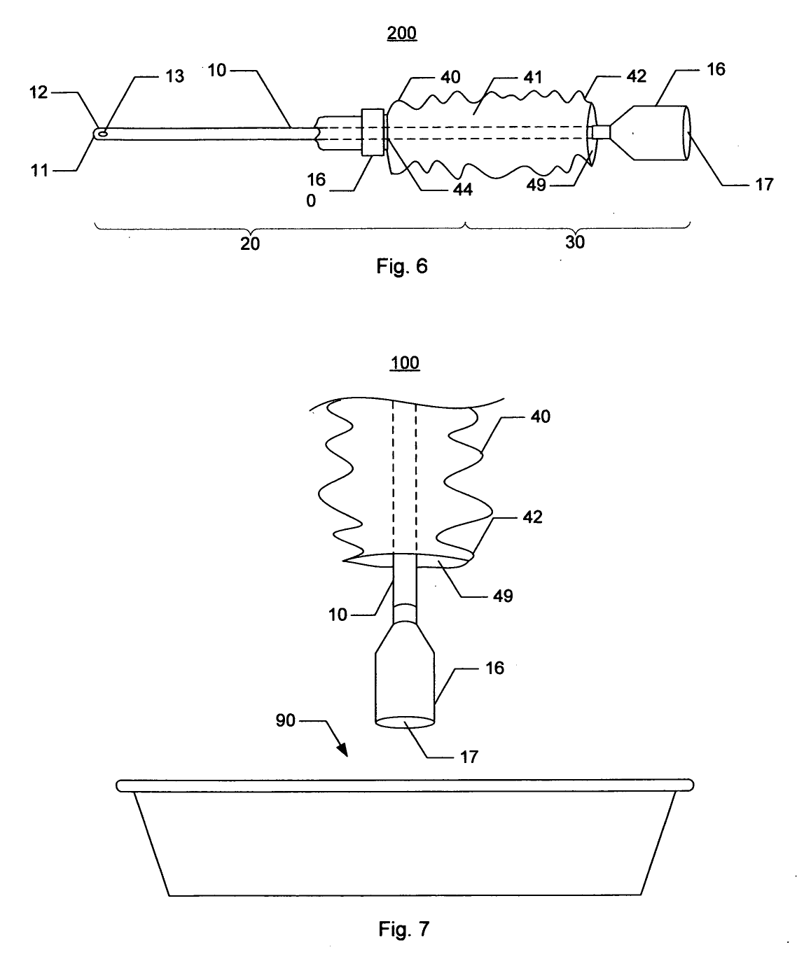

Referring to FIGS. 5 and 6, another embodiment of a sheathed catheter assembly 200 is illustrated. Assembly 200 is substantially equivalent to assembly 100 illustrated in FIG. 1, however, assembly 200 includes an introducer 160 that is different from introducer 60 illustrated in FIG. 1. Specifically, introducer 160 shown in FIGS. 5 and 6 is similar to an O'Neil® type tip with a “cross-cut” end. Embodiments of an O'Neil type tip are disclosed in U.S. Pat. No. 4,652,259, which is hereby incorporated herein by reference in its entirety.

In addition, in the embodiment illustrated in FIGS. 5 and 6, initially sheath 40 does not extend beyond second end 16 of catheter 10. Together, sheath 40 and introducer 160 completely enclose insertable portion 20, thereby specifically protecting insertable portion 20 from contamination. Use of catheter assembly 200 preferably occurs in a sterile field with the user handling device donning sterile gloves. However, in the event that contamination of the portions of catheter 10 extending though opening 49 (e.g., second end 16) occurs, such contamination is furthest from insertable portion 20 of catheter 10.

Referring to FIG. 7, an embodiment of sheathed catheter assembly 100 is positioned such that urine drained from the patient's bladder may exit outlet 17 of catheter 10 into a urine collection receptacle 90. Specifically, second end 16 is positioned such that urine flowing from outlet 17 is directed into urine collection receptacle 90. By grasping second end 16 and orienting the direction of outlet 17 without interference by sheath 40, the user of assembly 100 may easily direct the urine exiting outlet 17 into receptacle 90, a toilet, or wherever the user desires. In the embodiment illustrated in FIG. 3, urine collection receptacle 90 is a tray, however, in general, urine collection receptacle 90 may comprise any suitable device for capturing and holding urine drained from a patient's bladder including without limitation a collection tray, a collection bag, a toilet, etc.

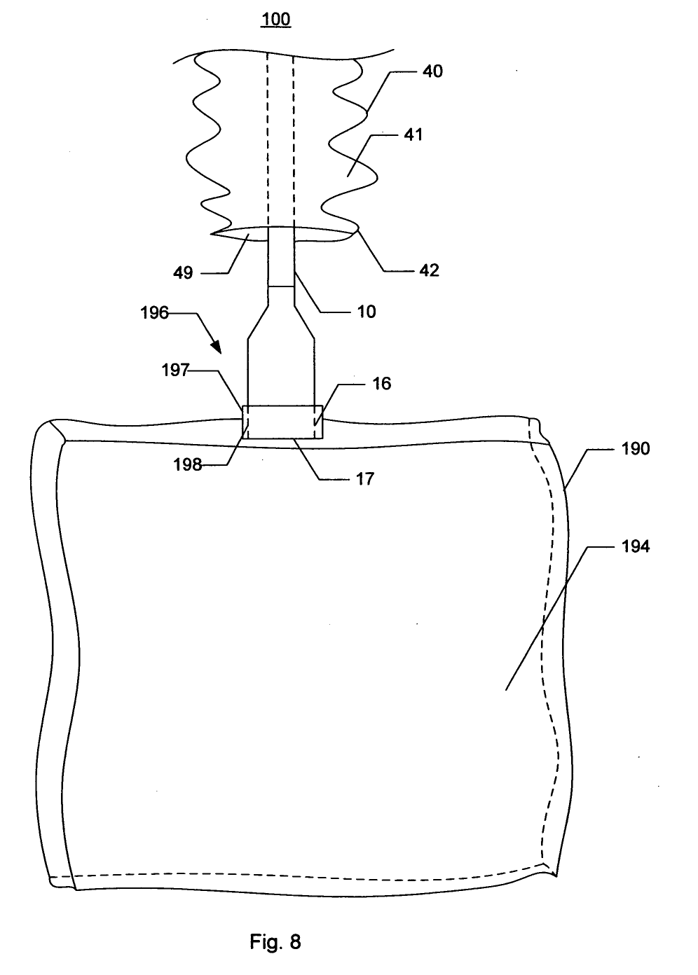

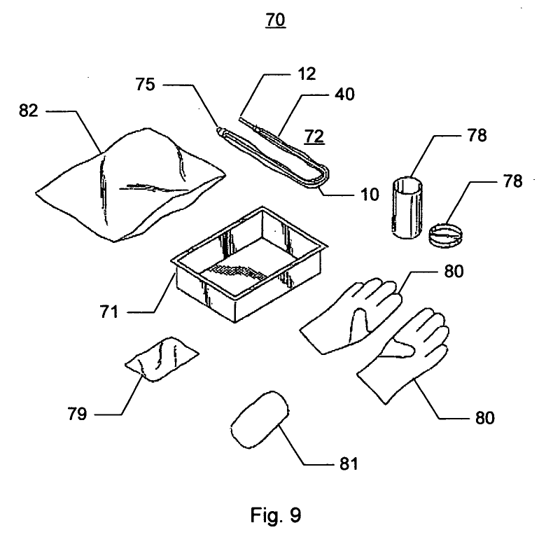

Referring to FIG. 8, an embodiment of sheathed catheter assembly 100 is coupled to a urine collection receptacle or bag 190. Urine collection bag 190 comprises an inner collection lumen 194 and a port assembly 196 that provides fluid access to collection lumen 194. Port assembly 196 is formed by a ring-shaped collar 197 having a through bore 198 in fluid communication with collection lumen 194. Collection receptacle 90 may be constructed of any suitable material. Collection receptacle 90 is preferably made of a flexible, water-proof material (e.g., plastic) and is sized to hold a volume of urine in the range of 700-2000 mL, preferably about 1000 mL.

Second end 16 of catheter 10 is coupled to port assembly 196 such that urine outlet 17 of catheter 10 is in fluid communication with bore 198 and collection lumen 194. Thus, urine may drain from the patient's bladder into urine inlet(s) 13, through catheter 10, out of urine outlet 17 and into collection lumen 194 of collection bag 190. Urine in collection bag 190 may be stored, sampled and tested, or disposed.

Second end 16 of catheter 10 may be coupled to port assembly 196 by any suitable means including without limitation heat sealing, adhesives, mating threads provided in bore 198 and on the outside of second end 16, a locking mechanism, or combinations thereof. Second end 16 may be fixed to port assembly 196 or releasably coupled to port assembly 196. In some embodiments, second end 16 is releasably coupled to port assembly 196 such that second end 16 may be disconnected from port assembly 196 and port assembly 196 may be used as a pour spout to take a sterile urine sample or to quickly empty collection bag 190.

Although port assembly 196 illustrated in FIG. 8 comprises a ring-shaped collar 197 having a bore 198 in fluid communication with collection lumen 194, in general, port assembly 196 with a bore 198 may have any suitable geometry that permits fluid flowing from urine outlet 17 of catheter 10 to be directed into collection lumen 194 of collection bag 190.

In light of the present disclosure, one of skill in the art would appreciate that some components of the embodiments of the sheathed catheter assemblies described herein (e.g., catheter assembly 100) could comprise any of a variety of configurations and appearances, as for instance, similar to those disclosed in U.S. Pat. No. 6,090,075 (House I), U.S. App. No. 60/708,893 (House II), U.S. application Ser. No. 11/326,699 (House III), and U.S. App. No. 60/781,225 (House IV). Further, some of the embodiments disclosed therein include possible variations in the configuration of urine collection receptacle 90, 190.

Catheterization Kit

Referring to FIG. 9, a representative catheterization kit (“cath kit”) 70 that is particularly useful for patient catheterizations includes a sheathed catheter assembly 72, representative of those shown and described above as sheathed catheter assemblies 100, 200. For example, sheathed catheter assembly 72 may include an introducer 60, 160, a catheter 10, and a sheath 40. The introducer end 44 of sheath 40 may be coupled to introducer 60, 160, if present. Further, sheath 40 may include an opening 49 in its free end 42 that may or may not extend beyond the second end 42 of sheath 40.

In addition to sheathed catheter assembly 72, the kit preferably also contains a packet of antiseptic swabs 79 (e.g., swabs, or the like, saturated with Betadine, Povidone-Iodine or other suitable antiseptic), disposable gloves 80, a small urine specimen bottle (with cap) 78, and a tray 71 that holds these supplies and also serves as a urine collection container (e.g., urine collection receptacle 90 illustrated in FIG. 7). Tray 71 preferably has a capacity in the range of 700-2000 mL, and more preferably about 1000 mL. In some embodiments, kit 70 may also include a urine collection bag similar (e.g., urine collection receptacle 190 illustrated in FIG. 8. Preferably, a fenestrated drape is also included in the kit. A gauze pad may also be included in the kit as a convenient wipe at the end of the procedure. The kit components are sealed within and protected by a sanitary wrapper or cover 82. All kit components are preferably disposable.

Preferably, with use of kit 70, the lubricant is provided within sheathed catheter assembly 72. For example, catheter 10 may be pre-lubricated with a sufficient amount of sterile lubricant present inside the sheath lumen. As another example, an introducer 60, 160 may include a lubricant reservoir that contains a lubricant that is applied to catheter 10 as catheter 10 is advanced through such introducer 60, 160.

Absent from the present kit, however, are the customary liquid antiseptic packet, cotton balls, tray for cotton balls, forceps, and packet containing lubricating jelly. In some embodiments, the fenestrated drape is also omitted from the kit without compromising sterile technique. The conventional waterproof absorbent pad is also unnecessary, as it was often included in the past primarily to provide a sterile field for placement of the sterile jelly. The above-mentioned items omitted from conventional catheterization trays are not needed for carrying out a streamlined catheterization procedure with the above-described sheathed catheter assembly 72 while maintaining sterile technique.

A drawback of conventional apparatus and methods is that touching the catheter to any surface outside of the sterile field, as when lubricating jelly is applied, for instance, increases the risk of contamination and urinary tract infection. In contrast, with the present kit, if the sheathed catheter assembly contacts any non-sterile surface, the insertable portion of the catheter remains sterile within the protective sheath. Another potential problem associated with conventional catheterization kits and procedures is that, if a glove touches anything outside the sterile field and then touches the catheter, the catheter becomes contaminated. If a sheathed catheter assembly is employed instead, then even if a one or both glove(s) becomes contaminated, the catheter nevertheless remains sterile inside the protective sheath. Thus, the risk of infection and patient morbidity may be reduced or eliminated with use of the new sheathed catheter assembly 72, catheterization kit 70, and catheterization procedure.

Catheterization Procedure

Embodiments of the sheathed catheter assemblies described herein (e.g., sheathed catheter assembly 100, 200) may be used alone or as part of catheterization kit 70 illustrated in FIG. 9. Referring to FIGS. 1-3, and using assembly 100 in this exemplary explanation, sheathed catheter assembly 100 is preferably employed as follows: the user grasps introducer 60 and/or catheter 10 through the soft, flexible sheath 40 near first end 11 of catheter 10 to align introducer 60 and tip 12 with the patient's urethra. In the case in which introducer 60 is an O'Neil® type tip, the projection of introducer 60 may be inserted about 1 to 2 cm into the urethra (or until the perpendicular flange prevents further insertion).

Still referring to FIGS. 1-3, first end 11 and tip 12 are advanced through introducer 60 and into the patient's urethra. As tip 12 and insertable portion 20 are advanced, introducer 60 and introducer end 44 of sheath 40 move towards second end 16. At about the same time, the user also slides free end 42 of sheath 40 toward second end 16. As a result, introducer end 44 and free end 42 of sheath 40 are generally moved towards each other and toward insertion stop location 15 as shown in FIG. 2. Continued advancement of insertable portion 20 coupled with continued sliding of free end 42 over second end 16 towards insertion stop location 15 results in gathering of sheath 40 between insertion stop location 15 and second end 16 of catheter 10 as shown in FIG. 3. Once second end 16 extends beyond opening 49, the user may directly grasp second end 16. By grasping introducer 60 with one hand and second end 16 with one hand, the user may continued to gently urge insertable portion 20 into the urethra and toward the bladder. Any pressure build-up within lumen 41 of sheath 40 due to gathering of sheath 40 is relieved through opening 49, thereby deterring some of the undesirable aspects of pressure build-up.

Referring to FIG. 9, and using sheathed catheter assembly 100 illustrated in FIG. 1 as an exemplary sheathed catheter assembly 72, catheterization kit 70 is preferably employed as follows: After opening the sanitary wrapper 82, the user (e.g., patient, nurse, or doctor) dons the gloves 80 and places the fenestrated drape, if provided, around the patient's genitalia. The packet 79 containing the antiseptic swabs is opened and the urethral area around the urethral opening is cleansed using the antiseptic swabs. Following proper sanitation, insertable portion 20 of sheathed catheter assembly 100 is advanced into the patient's urethra as described above in reference to FIGS. 1-3 When catheter tip 12 enters the patient's bladder a sufficient distance to commence draining of accumulated urine (e.g., about 1 cm), further insertion ceases.

Prior to commencement of urine drainage, second end 16 of catheter 10 is placed in fluid communication with tray 71 so that urine can drain into tray 71 while preventing the catheter from contacting the collected urine. Alternatively, any other suitable urine collection receptacle, such as urine collection bag 190 illustrated in FIG. 8, may be coupled to second end 16 of catheter 10 and used as an alternative to disposable tray 71. Specifically, the user may grasp second end 16 of catheter 10 to direct the flow of drained urine from outlet 17 into tray 71, or other collection receptacle if provided.

After commencement of urine flow, and if necessary, by grasping second end 16, outlet 17 of catheter 10 may be directed briefly into the specimen container 78, to collect a sterile specimen, as needed. Upon completion of urine evacuation, catheter 10 is withdrawn from the urethra and disposed of along with the other components of the kit. If desired, the catheter may be retracted into the sheath prior to disposal. The entire catheterization process can usually be accomplished by a nurse while maintaining sterile technique throughout the procedure.

The disclosed catheterization kit 70 and simplified catheterization procedure(s) offer a means to streamline patient catheterizations without compromising sterile technique. As compared to conventional “cath kits” or “cath trays”, every item that kit 70 eliminates potentially decreases the procedural steps and time required to conduct the catheterization. Fewer steps also reduces the patient's risk for urinary tract infection, and decreases inconvenience for the patient. Further, by eliminating certain items typically included in conventional “cath kits” or “cath trays”, new kit 70 described herein offers the potential for reducing the costs of catheterizations.

In the manner described, embodiments described herein are intended to offer the potential to improve the convenience and sterility for catheterization procedures. In addition, some embodiments are intended to reduce the costs of catheterization procedures. For instance, as compared to conventional catheterizations, some embodiments may reduce the nursing time required to perform the catheterization, reduce the cost of the sheathed catheter assembly (e.g., reduce manufacturing costs), and/or reduce the cost of the “cath kit” employed to perform the catheterization. Still further, some embodiments described herein are designed to deter pressure build-up within the lumen of a protective sheath surrounding the catheter, thereby preventing some of the problems associated with pressure build-up within the sheath of a sheathed catheter assembly.

The disclosures of all patents, patent applications, and publications cited herein are hereby incorporated herein by reference in their entirety, to the extent that they provide exemplary, procedural, or other details supplementary to those set forth herein. Further, the discussion of a reference in this disclosure is not an admission that it is prior art to the present invention, especially any reference that may have a publication date after the priority date of this application.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A urinary catheter assembly comprising:
a catheter including a first end having a urine inlet, a second end having a urine outlet, and a urethra-insertable portion;
an introducer adapted to slidingly engage the catheter, wherein the first end of the catheter is at least partially disposed within the introducer; and
a pliable sheath comprising a lumen, an introducer end coupled to the introducer, and a free end opposite the introducer end, the introducer and sheath together enclosing all of the urethra-insertable portion;
wherein the free end includes an opening in fluid communication with the lumen and an environment outside the lumen;
wherein the opening has a size sufficient to permit the second end of the catheter to pass therethrough.

2. The catheter assembly of claim 1, wherein the free end of the sheath is adapted to extend beyond the second end of the catheter.

3. The catheter assembly of claim 1, wherein the catheter further comprises a non-insertable portion extending from the insertion stop location to the second end, wherein at least part of the non-insertable portion comprises an uncovered region extending through the opening in the free end of the sheath.

4. The catheter assembly of claim 1, wherein the catheter has a total length from the first end to the second end, wherein the urethra-insertable portion represents at least half of the total length of the catheter.

5. The catheter assembly of claim 1, further comprising a urine collection receptacle coupled to the second end of the catheter.

6. The catheter assembly of claim 5, wherein the urine collection receptacle includes a lumen in fluid communication with the outlet of the catheter.

7. The catheter assembly of claim 1, wherein the introducer further comprises a reservoir for storing a lubricant.

8. The catheter assembly of claim 1, wherein at least the urethra-insertable portion is pre-lubricated with a lubricant.

9. A urinary catheter assembly comprising:
a catheter comprising a first end having a urine inlet, a second end having a urine outlet, and a urethra-insertable portion, wherein the second end has an outer perimeter;
a pliable sheath comprising a lumen and enclosing at least a part of the insertable portion, wherein the sheath has a free end adapted to extend beyond the second end of the catheter;
wherein the free end of the sheath includes an opening having an inner surface distance; and
wherein the inner surface distance of the opening is greater than the outer perimeter of the second end.

10. The catheter assembly of claim 9, wherein the second end of the catheter is adapted to extend beyond the free end of the sheath through the opening in the free end of the sheath.

11. The catheter assembly of claim 9, further comprising an introducer that slidingly engages the catheter, wherein the sheath includes an introducer end coupled to the introducer.

12. The catheter assembly of claim 11, wherein the introducer includes a lubricant reservoir containing a lubricant.

13. The catheter assembly of claim 11, wherein the introducer and sheath together enclose all of the urethra-insertable portion of the catheter.

14. A catheterization kit comprising:
the urinary catheter assembly of claim 1; and
a sanitary wrapper enclosing the assembly.

15. The kit of claim 14 consisting essentially of the following items:
the urinary catheter assembly;
at least one antiseptic swab;
disposable gloves;
a urine specimen container;
a tray capable of holding the catheter assembly, the at least one swab, the gloves, and the specimen container, wherein the tray is also capable of serving as a urine collection receptacle; and
a sanitary wrapper enclosing the kit.

16. The kit of claim 15, excluding one or more drapes.

17. The kit of claim 16 excluding forceps.

18. The kit of claim 17 excluding antiseptic liquid.

19. A catheterization kit comprising:
the urinary catheter assembly of claim 8; and
a sanitary wrapper enclosing the assembly.

20. A urinary catheter assembly comprising:
a catheter including a first end having a urine inlet, a second end having a urine outlet, and a urethra-insertable portion;
an introducer adapted to slidingly engage the catheter, wherein the first end of the catheter is at least partially disposed within the introducer;
a pliable sheath comprising a lumen, an introducer end coupled to the introducer, and a free end opposite the introducer end, the introducer and sheath together enclosing all of the urethra-insertable portion;
wherein the free end includes an opening in fluid communication with the lumen and an environment outside the lumen;
wherein the opening has a size sufficient to permit the second end of the catheter to pass therethrough; and
a urine collection bag comprising a collection lumen and a port assembly in fluid communication with the collection lumen, wherein the second end of the catheter is coupled to port assembly with the urine outlet in fluid communication with the collection lumen.

21. The catheter assembly of claim 20, wherein the second end of the catheter is integral with the port assembly.

22. The catheter assembly of claim 21, wherein the second end of the catheter is releasably coupled to the port assembly.

23. The catheter assembly of claim 22, wherein the port assembly is operable to serve as a urine pour spout when the second end of the catheter is separated from port assembly of the collection bag.

24. A catheterization kit comprising:
the urinary catheter assembly of claim 20; and
a sanitary wrapper enclosing the assembly.
